(19) United States
(12) Patent Application Publication
(10) Pub. No.: US 2019/0244714 A1
(43) Pub. Date: Aug. 8, 2019

(54) SICKNESS PREDICTION APPLICATION SYSTEM

(71) Applicant: DAPAPULT, INC., Aurora (CA)

(72) Inventors: Michael MORRA, Aurora (CA); Tony PETICCA, Aurora (CA)

(73) Assignee: DAPAPULT, INC., Aurora, ON (CA)

(21) Appl. No.: 16/343,193

(22) PCT Filed: Oct. 20, 2017
(86) PCT No.: PCT/CA2017/051257

§ 371 Date: Apr. 18, 2019

Related U.S. Application Data
(60) Provisional application No. 62/410,819, filed on Oct. 20, 2016.

(51) Int. Cl. G16H 50/30; G16H 50/70; G06N 7/00; G06F 17/15

(52) U.S. Cl. CPC G16H 50/30; G06F 17/15; G06N 7/005; G16H 50/70

(57) ABSTRACT

A method and system for predicting the probability of sickness, the method comprising establishing a communication link between a sickness prediction application and a health tracking device having one or more measurement devices or sensors; inputting user symptoms utilizing the user device at one or more sickness timepoints; obtaining sensor or detection data from the health sensor at the sickness timepoint; generating sickness models, wherein the sickness model is a correlation of the user symptoms and the sensor data at the sickness timepoint; obtaining new sensor data from the health sensor at predetermined or other intervals; and comparing the new sensor data with the sickness model to predict the probability of the user having or developing an acute, new or recurring sickness.

### CROSS REFERENCE TO RELATED APPLICATION

This application claims the priority of U.S. Provisional Application No. 62/410,819, entitled “SICKNESS PREDICTION APPLICATION SYSTEM,” filed on Oct. 20, 2016, the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to a sickness prediction system, and in particular, collecting data from a health tracking device and utilizing the collected data to identify and predict new or recurring illnesses.

### Description of the Related Art

Wearable devices, such as from Fitbit®, Garmin®, or Apple®, have been developed to help individuals monitor and visualize their fitness levels and daily exercise routines. These devices can measure or monitor a user's attributes, such as the user's heart rate, number of steps taken, distance traveled, calories burned, speed at which the user is moving, etc. The wearable device may present information about the user's attributes on a display. Users who are most interested in these devices tend to be active individuals and/or athletes that regularly participate in a sport of some sort, such as running, cycling, basketball, swimming, etc. Fitness trackers often help these types of individuals track their progress on certain fitness-related goals. For example, some users may use a fitness tracker to help monitor their running pace or distance traveled in a race.

### SUMMARY OF THE INVENTION

The present invention provides a system for analyzing data from a health tracking device. The system comprises a memory device having executable instructions stored therein, and a processing device, in response to the executable instructions, operative to: receive user symptoms associated with a user of the health tracking device at a sickness timepoint; retrieve health sensor data of the health tracking device at a sickness timepoint; generate a sickness model, wherein the sickness model includes a correlation of the user symptoms and the health sensor data at the sickness timepoint; retrieve real-time health sensor data from the health tracking device at predetermined intervals; and compare the real-time health sensor data with the sickness model; and predict the probability of the user having a recurring sickness based on the comparison.

The health sensor data may include a measurement selected from the group consisting of: heart rate, pulse rate, galvanic skin response, sleep, blood oxygen level, body temperature, pulse, activity, exercise, nutrition, calories, hydration, motion intensity, perspiration, heat flux, and environmental conditions. The system may further comprise the processing device configured to retrieve user attributes of the user, the user attributes including sex, age, height, and weight of the user, and calibrate the sickness model based on the user attributes. The health tracking device may include at least one of fitness trackers, wrist bands, bracelets, watches, rings, pendants, jewelry, implants, radio-frequency identification devices. In one embodiment, the user symptoms may include an indication of sickness. The user symptoms may include at least one of headaches, cough, fever, chest congestion, runny nose, nasal congestion, fatigue, fever, aches, pains, sore throat, sneezing, watery eyes, malaise, chills, nausea, vomiting, and diarrhea. The system may further comprise the processing device configured to calculate a similarity of the real-time health sensor data to the sickness model.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates a computing system according to an embodiment of the present invention.

FIG. 2 illustrates a computing system according to another embodiment of the present invention.

FIG. 3 illustrates a flowchart of a method for predicting sickness according to an embodiment of the present invention.

FIG. 4 illustrates a data flow diagram of a system for classifying sick data according to an embodiment of the present invention.

FIG. 5 illustrates an exemplary interface for entering symptoms according to an embodiment of the present invention.

FIG. 6 illustrates an exemplary interface for presenting a sickness probability score according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

The present application discloses a sickness prediction system for use with a user device, including mobile devices, such as smartphones and tablets, and non-mobile devices, such as desktop computers, optionally having a display screen and network connectivity. The system may collect data from wearable or non-wearable monitoring devices, such as fitness trackers, wrist bands, bracelets, watches, rings, pendants, jewelry, implants, radio-frequency identification (RFID) device, or other devices optionally connected by Internet, Bluetooth, NFC or WiFi, and then utilizes the collected data to recognize health patterns to identify and predict new or recurring illnesses. A sickness prediction application may be used in conjunction with the user device to facilitate the entry of user symptoms associated with a user of the device at a sickness timepoint. The application may have network connectivity for downloading sensor data from a sensor at a sickness timepoint, and may be operative to generate calibrated sickness models, wherein the calibrated sickness models are based on a correlation of the user's symptoms and the sensor data at the sickness timepoint. The application may also download new sensor data from the sensor at predetermined or other intervals. In one embodiment, the application may be operative to compare the new sensor data with the calibrated sickness models to predict the probability of the user having a recurring sickness.

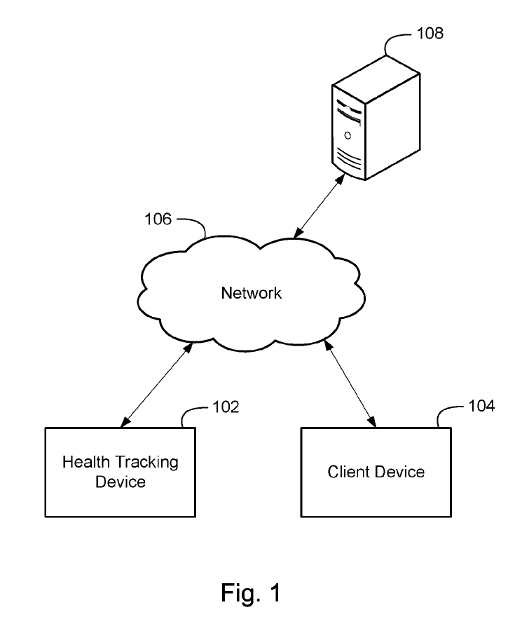

FIG. 1 illustrates a computing system according to an embodiment of the present invention. The computing system presented in FIG. 1 includes health tracking device 102, client device 104, network 106, and analysis server 108. Health tracking device 102 may comprise computing devices having a central processing unit and memory unit capable of connecting to a network. The health tracking device 102 may optionally include, for example, a display screen, network connectivity, sensors, motion and location identifying technology, GPS, temperature and moisture monitors, pulse and pulse oximetry monitors, environmental sensors and other measurement and detection devices. According to one embodiment, health tracking device 102 may comprise an implant (body) including one or more sensors configured to measure certain biometric data and network connectivity for transmitting the biometric data to analysis server 108. The health tracking device 102 may communicate with analysis server 108 via network 106 for transmitting sensor data at any given timepoint. The health tracking device 102 may include or may execute a variety of possible applications, such as a client software application enabling communication with other devices, such as communicating one or more messages, such as via email, short message service (SMS), or multimedia message service (MMS), including via a network, as well as a social network, including, for example, Facebook, LinkedIn, Twitter, Pinterest, Snapchat, Instagram, or Google+, to provide only a few possible examples.

Client device 104 may comprise computing devices (e.g., desktop computers, television devices, terminals, laptops, personal digital assistants (PDA), cellular phones, smartphones, tablet computers, e-book readers, or any computing device having a central processing unit and memory unit capable of connecting to a network). Client devices may also comprise a graphical user interface (GUI) or a browser application provided on a display (e.g., monitor screen, LCD or LED display, projector, etc.). According to one embodiment, the client device 104 may include or execute a sickness prediction application. The sickness prediction application may facilitate the entry of symptoms associated with a user of the health tracking device at time points of sickness or ailment. In an alternative embodiment, the sickness prediction application may be installed on health tracking device 102 where the user may indicate symptoms of sickness or ailment directly from the health tracking device 102.

The analysis server 108 may be operable to generate calibrated sickness models wherein the calibrated sickness models include a measure of the probability of the user becoming sick within a given time frame using, for example, a correlation of the user's reported symptoms and the sensor data at one or more sickness timepoints. The analysis server 108 may download new sensor data from health tracking device 102 at predetermined or other intervals, and may be operative to compare the new sensor data with the calibrated sickness models to predict the probability of the user having or developing an acute, new or recurring sickness, or alerting a user of changes in their baseline health measurements which may suggest chronic illnesses such as cancers, metabolic diseases, cardiovascular issues, or other serious health issues that may be helped through early detection. In one or more embodiments, the analysis server 108 may utilize the user's data-including, for example, sensor and input data-and the calibrated sicknesses models over the course of time to implement changes to the sickness prediction application's ability to better predict future sicknesses in the user with greater accuracy. A sick score may be generated from the probability determined by the analysis server 108 and present the score to the user through the sickness prediction application on client device 104, or on health tracking device 102 in certain embodiments.

Network 106 may be any suitable type of network allowing transport of data communications across thereof. The network 106 may couple devices so that communications may be exchanged, such as between servers and client devices or other types of devices, including between wireless devices coupled via a wireless network, for example. A network may also include mass storage, such as network attached storage (NAS), a storage area network (SAN), cloud computing and storage, or other forms of computer or machine-readable media, for example. In one embodiment, the network may be the Internet, following known Internet protocols for data communication, or any other communication network, e.g., any local area network (LAN) or wide area network (WAN) connection, cellular network, wire-line type connections, wireless type connections, or any combination thereof. Communications and content stored and/or transmitted to and from health tracking devices may be encrypted using, for example, the Advanced Encryption Standard (AES) with a 128, 192, or 256-bit key size, or any other encryption standard known in the art.

Servers, as described herein, may vary widely in configuration or capabilities but are comprised of at least a special-purpose digital computing device including at least one or more central processing units and memory. A server may also include one or more of mass storage devices, power supplies, wired or wireless network interfaces, input/output interfaces, and operating systems, such as Windows Server, Mac OS X, Unix, Linux, FreeBSD, or the like.

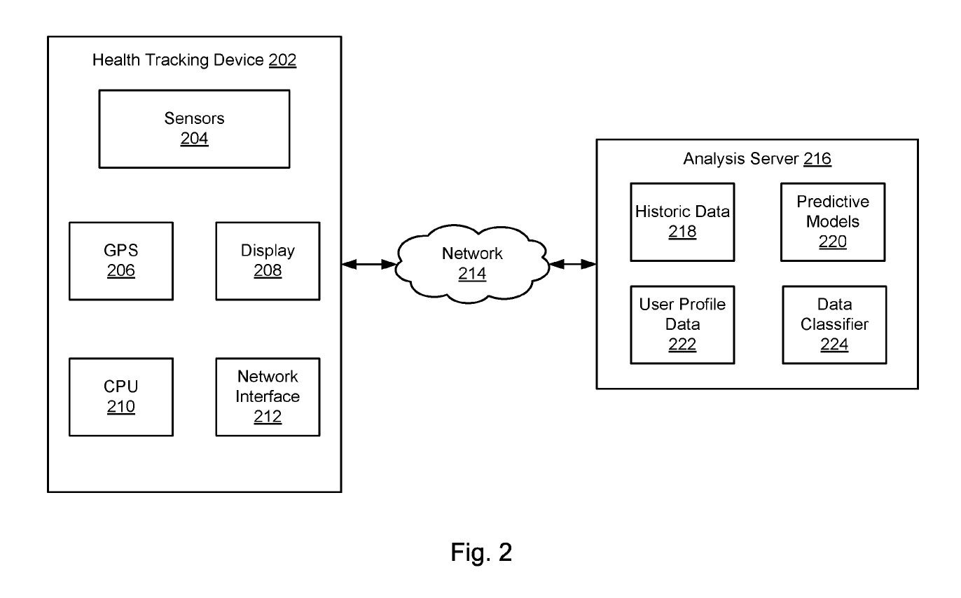

FIG. 2 presents a system that includes an analysis server 216 that can use historic data 218 to generate one or more predictive models 220. For instance, the historic data 218 may include a collection of readings from sensors 204 such as, electrocardiography (ECG) for sensors for heart rate (e.g., minute, average, resting, active), sleep sensors to detect, e.g., rapid eye movement (REM sleep), sleep/awake duration, disturbance, movement), and pulse oximeter/photoplethysmogram (PPG) sensors for blood oxygen readings, as well as sensors for detecting body temperature, blood pressure, blood sugar levels, weather, barometric pressure, humidity, pollen count, and smog. Analysis server 216 may create the historic data 218 by archiving data from sensors 204 for a duration of time (e.g., days, weeks, months, years). Additionally, historic data 218 may import sensor data from third-party services (not illustrated) including sensor data from the health tracking device 202, e.g., from a product manufacturer or a native service offering. The analysis server 216 may also receive user profile data 222 for a particular user and provide the user profile data 222 to data classifier 224. For example, the analysis server 216 may receive the user profile data 222 from a combination of the health tracking device 202, another client device, e.g., a desktop or a laptop, or both.

The user profile data 222 may include personal data, such as a user's sex. The user profile data 222 may include biometric data, such as a user's age, weight, height, blood pressure, total cholesterol, HDL cholesterol, or other appropriate biometric data. The user profile data 222 may include health behavior data, such as a total number of minutes of exercise per week, an intensity level for exercise, e.g., overall or for particular exercise activities, whether and how much a user smokes, or other appropriate health behavior data. The analysis server 216 may create predictive models 220 that receive, as input, user attributes such as sex, age, whether the user smokes, whether and how much the user exercises, body mass index, blood pressure, total cholesterol, high-density lipoprotein (HDL) level, chronic disease diagnosis, or a combination thereof. The user profile data 222 may be generated from survey data, data measured by sensors 204, or a combination. In some examples, the user profile data 222 may be data from a biometric screening, a health risk assessment, a questionnaire, or a combination thereof.

In some embodiments, the analysis server 216 may determine relationships between various metrics/sensor measurements, user attributes, and sickness or ailments to calibrate the predictive model. For example, the analysis server 216 may train predictive models (supervised machine learning) using particular data sets from historic data 218 that correspond to a time point when the user reports sickness or ailments (labeling sick data) and learn a relationship of a reported sickness on the health metrics of the user. The learned relationship may be further calibrated with the user attributes. The analysis server 216 may use data representing the relationships during predictive model generation, run-time use of the predictive model, or both. In some implementations, the analysis server 216 may generate different predictive models for each user according to user profile data 222. The data classifier 224 applies the user profile data 222 to one of the predictive models, e.g., specific to the particular user, the user profile data 222, or both. For instance, the data classifier 224 may select a particular predictive model from the predictive models 220 based on the types of user profile data 222 for the user, e.g., specific numerical values, numerical ranges, no numerical data, whether disease or family medical history data is available, etc., or based on particular user attribute data for the user, e.g., age, sex, or both. In some examples, the data classifier 224 may select the particular predictive model based on both the types of user profile data 222 for the user and particular user profile data 222 for the user.

The analysis server 216 may combine an output of one or more predictive models to determine an overall health risk score for the user. When the overall health risk score satisfies a threshold value, the data classifier 224 may assign a low risk profile to the user. The low risk profile may indicate that the user has few, if any, attributes which they can improve. The data classifier 224 may also determine attribute scores for user attributes to determine which specific attributes the user can improve. When the overall score does not satisfy the threshold value, the data classifier 224 may determine whether the overall score belongs to a medium or high-risk profile. In some implementations, the data classifier 224 may determine an attribute score for a user likelihood of dying because of a particular disease, an attribute score for a user likelihood of developing a disease or a particular disease, or both. For instance, the data classifier 224 may use a predictive model and user attribute data to determine a score that is indicative of the user developing a particular disease. The data classifier 224 may use the score to determine task recommendations to improve the score, e.g., to reduce the likelihood that the user will develop the particular disease.

According to an alternative embodiment, the analysis server 216 may utilize unsupervised machine learning to infer sick data from an unlabeled data set in historic data 218. An unsupervised machine learning model may determine sickness susceptibility based on subtle changes in the health metrics collected without the user labeling or calibrating sick data. For example, cluster analysis may be used for exploratory data analysis to find hidden patterns or grouping in the unlabeled data set. Cluster analysis may include grouping data in such a way that objects in the same group (a cluster) are more similar to each other than those in other groups. Clustering algorithms such as hierarchical clustering, K-means clustering, Gaussian mixture models, and Hidden Markov models, may be used to perform clustering of the unlabeled data set. In another example, anomaly detection (or outlier detection) may be used to identify data from the unlabeled data set which do not conform to an expected pattern or other items in the unlabeled data set.

The data classifier 224 may provide a user's overall score to health tracking device 202 on display 208 or a separate client device for presentation to a user. Health tracking device 202 includes a central processing unit (CPU) 210. When the health tracking device 202 presents content on the display 208, the health tracking device 202 may receive user input from a touch screen surface included in the display 208, hard or soft buttons included in the health tracking device 202, or any other appropriate input. The health tracking device 202 receives data from one or more of the sensors 204 and provides the data to the analysis server 216. Data from the one or more sensors 204 may also be presented for display to the user on display 208. The health tracking device 202 may include a global positioning system (GPS) 206 that monitors a physical location of the health tracking device 202.

The health tracking device 202 and the analysis server 216 may collect data in response to receipt of user input indicating a user's permission to collect data. For instance, when first used, the user may be prompted for permission to collect and analyze attribute data for the user. The health tracking device 202 may send and receive data over a network 214 using network interface 212. The network 214, such as a local area network (LAN), wide area network (WAN), the Internet, or a combination thereof, connects the health tracking device 202 and the analysis server 216.

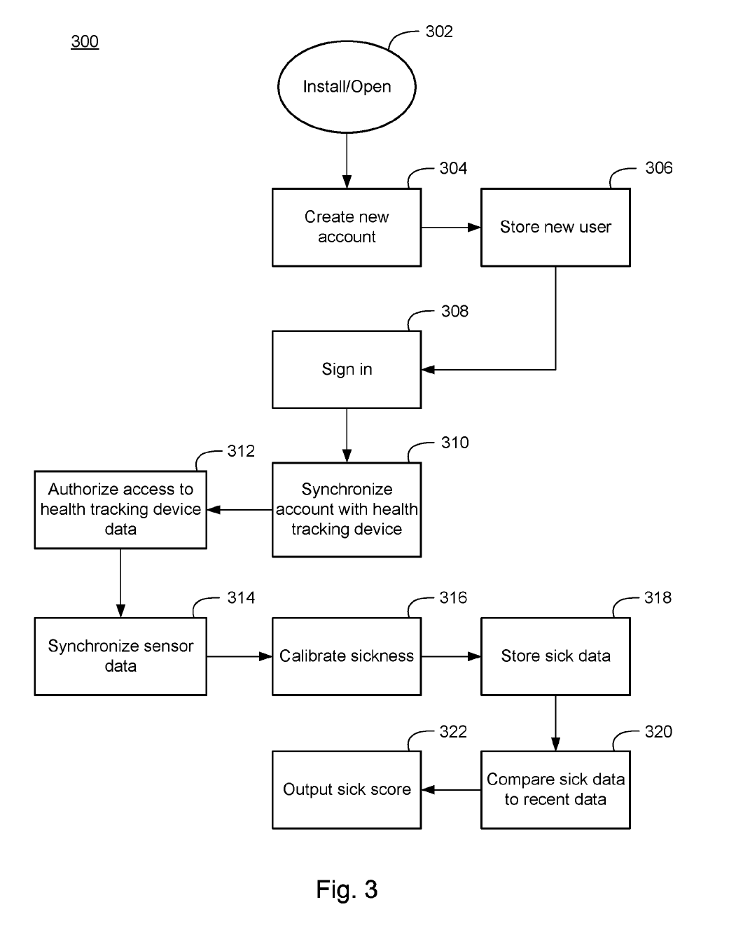

FIG. 3 presents an illustration of a flow chart schematic of an exemplary embodiment of a method for predicting sickness. A method 300 is shown schematically in FIG. 3. A sickness prediction application may be installed or opened on a user client device, step 302. The sickness prediction application may comprise a client interface to an analysis server for analyzing sensor data. A user can install the sickness prediction application from, for example, an app market place onto a user device. The user device, such as a tablet, smartphone, other mobile device or a stationary computer, may have location identifying technology, a display screen and network connectivity, among other features. The user device may further include sensors, motion and location identifying technology, GPS, temperature and moisture monitors, pulse and pulse oximetry monitors, environmental sensors, and other measurement and detection devices. The environmental sensors may also measure (or retrieve data from a server) the specific weather conditions, such as pollen count, smog, temperature and humidity, based on the location of the user. The environmental sensor may also measure the ambient temperature in the user's immediate area. The sickness prediction application may be installed on user devices utilizing an operating system, such as Android, iOS, Windows, Blackberry, Firefox OS, Sailfish OS, Tizen, Ubuntu Touch OS, and H5OS, among others.

A new user account for the sickness prediction application is created, step 304. The user may enter the user's personal information, which may include a login name, password, name, sex, age, height, weight, blood type, and other metrics and identifying information. The new user account is stored in a database associated with the sickness prediction application (at the analysis server), step 306. A sign in is received for using the sickness prediction application with the new user account, step 308.

The user account is synchronized with a health tracking device, step 310. A health tracking device may include different types of health sensors such as, wearable or non-wearable monitoring devices, such as fitness trackers, wrist bands, bracelets, watches, rings, pendants, jewelry, implants, RFIDs, or other devices optionally connected by Internet, Bluetooth, NFC or WiFi. The sickness prediction application may be utilized with health sensors from Fitbit®, Jawbone®, Garmin®, Motorola Mobility®, Basis®, BodyMedia®, among others. Health sensors may include display screens, sensors, motion and location identifying technology, GPS, temperature and moisture monitors, pulse and pulse oximetry monitors, environmental sensors, and other measurement and detection devices. The environmental sensors may measure the specific weather conditions, such as pollen count, smog, temperature and humidity, based on the location of the user. The environmental sensor may also measure the ambient temperature in the user's immediate area.

Authorization is received for allowing the sickness prediction application access to sensor data from the user's sensor data account, step 312. The user's sensor data account may include an aggregation of sensor data from health tracking device in addition to sensor data that may be uploaded from the user's health tracking device to third-party services or native service offerings. The sensor can track health data, including activity, exercise, nutrition, calories, weight, hydration, sleep, heart rate, pulse rate, galvanic skin response, motion intensity, body temperature, perspiration, heat flux, location of the user, blood oxygen levels, environmental conditions (e.g., pollen count, smog, temperature, humidity, ambient temperature in the user's immediate area), and other measurements. The user's sensor data account is accessed by the analysis server and the sensor data is synchronized to the analysis server via the sickness prediction application, step 314.

A sickness calibration is received, step 316. The user may navigate to a sickness calibration screen and generate calibrated sickness models by explicitly or implicitly indicating that he/she is sick, along with the symptoms they are experiencing. For example, when the user feels or begins to feel sick, unwell, or generally different than normal, the user may navigate to a “Sickness Calibration” screen of the sickness prediction application to input data by entering symptoms that they are experiencing. Symptoms may include, but are not limited to, headaches, cough, fever, chest congestion, runny nose, nasal congestion, fatigue, fever, aches, pains, sore throat, sneezing, watery eyes, malaise, chills, nausea, vomiting, diarrhea, among others.

Sick data is gathered, step 318. The analysis server may classify and store sensor data that coincides with a period of a reported sickness (e.g., before, during, and after entering of symptoms) as sick data and is used to train a particular calibrated sickness model. The sick data is compared to recent data, step 320. The analysis server may perpetually or retrospectively compare historical sensor data, at predetermined or other intervals, with, for example, hourly, daily, or bi-daily, the most recent sensor data (e.g., real-time) from the health tracking device against the sick data in the calibrated sickness models using a data similarity algorithm. The data similarity algorithm may calculate the similarity or differences of the most recent data to the sick data in the calibrated sickness models and classify/predict the most recent data recorded from the health sensor as potentially sick data. The potential of user sickness may be calculated using trends and patterns between the most recent data and the calibrated sickness models. In one embodiment, the analysis server may generate predictive models to determine likelihoods that a user will get cancer, diabetes, sleep apnea, or another chronic disease.

The sickness prediction application may output a sick score based on one or more of the calibrated sickness models, step 322. A higher score may indicate a greater chance of sickness based on similarities between the calibrated sickness models and the user's recent data. The sickness prediction application may also provide the user with daily recommendations on how to prevent any future sickness based on the sick score. According to one embodiment, a user may be alerted of changes in their baseline health measurements which may suggest chronic illnesses such as cancers, metabolic diseases, cardiovascular issues, or other serious health issues that may be helped through early detection.

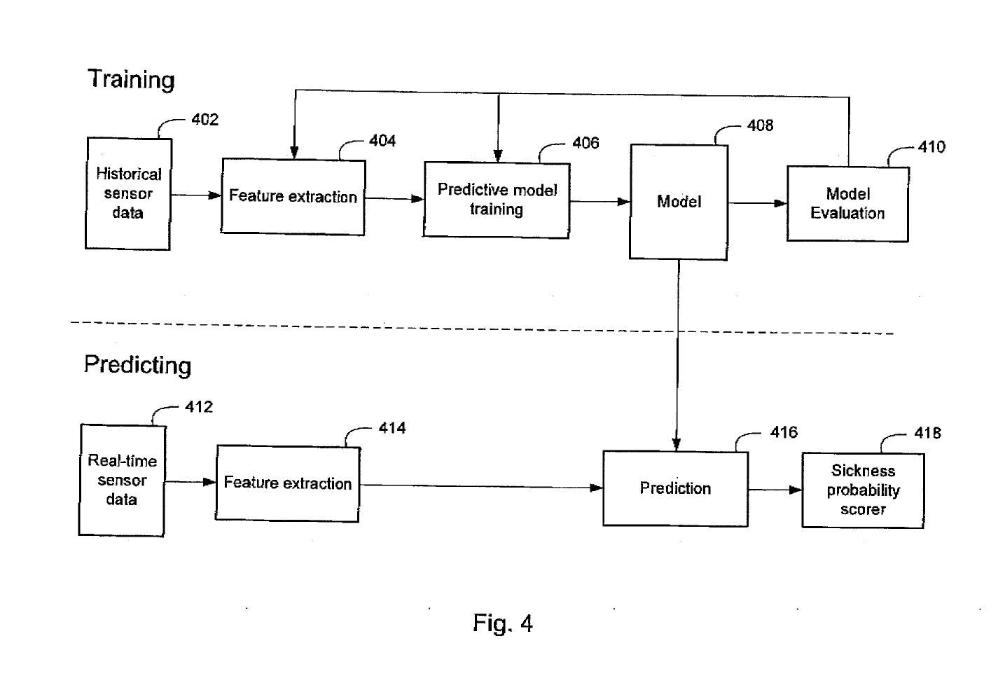

FIG. 4 presents a data flow diagram of a system for classifying sick data according to one embodiment of the present invention. Training a model may include retrieving historical sensor data 402 from a health tracking device. A feature extraction module 404 may extract features from the historical sensor data. The features may include key health metrics that are determined important for certain calibrated sickness models. For example, body temperature and resting heart rate may be required for training a calibrated sickness model for influenza.

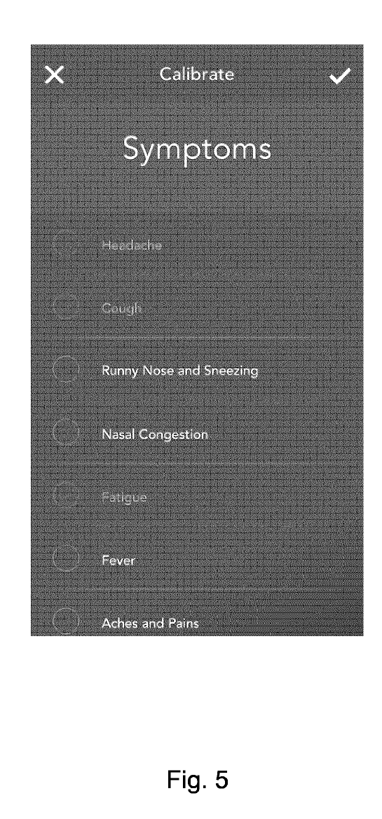

A predictive model training module 406 may train a predictive model by via calibration of the historical sensor data by the user of the health tracking device. The user may calibrate the historical sensor data by entering various symptoms to identify sick data. The predictive model training module 406 may take a snap shot of one or more health metrics and classify the data leading up to the calibration (entering of symptoms) as sick data. FIG. 5 presents an exemplary interface for entering symptoms.

Referring back to FIG. 4, a model 408 is generated by the predictive model training module based on the calibration of sick data. The model 408 may be evaluated by model evaluation module 410. According to one embodiment, model evaluation module 410 may receive feedback associated with the accuracy of the model 408. The feedback may include user confirmation of output provided by model 408 (e.g., sickness or health risk scoring). The feedback may be used by model evaluation module 410 to cause feature extraction module 404 to re-examine the features of historical sensor data 402 and update model 408 by predictive model training module 406.

Real-time sensor data 412 may be retrieved from the health tracking device to predict the probability of sickness. The real-time sensor data 412 may be read from the health tracking device at predetermined intervals, for example, a daily basis. Feature extraction module 414 may extract features (e.g., health metrics) from the real-time sensor data and forward the features to prediction module 416. Prediction module 416 may compare the real-time sensor data against the sick data from model 408. By calculating the similarity in data trends from the “unclassified data” of the real-time sensor data 412 against the calibrated sick data of the model 408, the prediction module 416 can determine if the data being read in real-time can be also classified as sick data.

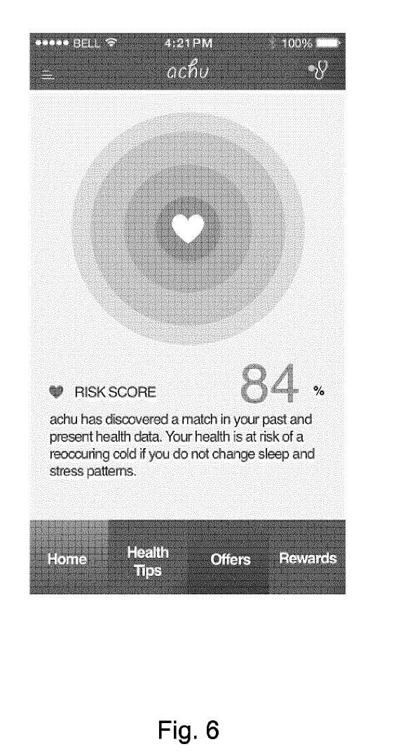

Sickness probability scorer 418 may generate a real-time sickness probability score. FIG. 6 presents an exemplary interface for presenting a sickness probability score. The sickness probability score may be based on a match in sick data from model 408 with the real-time sensor data 412. In one embodiment, the higher the similarity between the data, the higher the probability the user may have an illness. That is, if the sick data in model 408 is near identical to the real-time sensor data 412, the user may be labelled as a high probability candidate of an illness.

FIGS. 1 through 6 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps). In software implementations, computer software (e.g., programs or other instructions) and/or data is stored on a machine-readable medium as part of a computer program product, and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random-access memory (RAM); a read only memory (ROM); a removable storage unit (e.g., a magnetic or optical disc, flash memory device, or the like); a hard disk; or the like.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A system for analyzing data from a health tracking device, the system comprising:
a memory device having executable instructions stored therein; and
a processing device, in response to the executable instructions, configured to:
receive user symptoms associated with a user of the health tracking device at a sickness timepoint;
retrieve health sensor data of the health tracking device at a sickness timepoint;
generate a sickness model, wherein the sickness model includes a correlation of the user symptoms and the health sensor data at the sickness timepoint;
retrieve real-time health sensor data from the health tracking device at predetermined intervals; and
compare the real-time health sensor data with the sickness model; and
predict the probability of the user having a recurring sickness based on the comparison.

2. The system of claim 1 wherein the health sensor data includes a measurement selected from the group consisting of: heart rate, pulse rate, galvanic skin response, sleep, blood oxygen level, body temperature, pulse, activity, exercise, nutrition, calories, hydration, motion intensity, perspiration, heat flux, and environmental conditions.

3. The system of claim 1 further comprising the processing device configured to:
retrieve user attributes of the user, the user attributes including sex, age, height, and weight of the user; and
calibrate the sickness model based on the user attributes.

4. The system of claim 1 wherein the health tracking device includes at least one of fitness trackers, wrist bands, bracelets, watches, rings, pendants, jewelry, implants, radio-frequency identification devices.

5. The system of claim 1 wherein the user symptoms include an indication of sickness.

6. The system of claim 1 wherein the user symptoms include at least one of headaches, cough, fever, chest congestion, runny nose, nasal congestion, fatigue, fever, aches, pains, sore throat, sneezing, watery eyes, malaise, chills, nausea, vomiting, and diarrhea.

7. The system of claim 1 further comprising the processing device configured to:
calculate a similarity of the real-time health sensor data to the sickness model.
